(19) United States
(12) Patent Application Publication
(10) Pub. No.: US 2006/0088528 A1
(43) Pub. Date: Apr. 27, 2006

(54) Methods and compositions for treating alzheimer's disease

(76) Inventors: Bradley T Hyman, Swampscold, MA (US); Dudley Strickland, Brookeville, MA (US); Ayae Kinoshita, Sakyo-ku (JP); Christa Whelan, Charlestown, MA (US); G William Rebeck, Washington, DC (US)

Correspondence Address: WOLF GREENFIELD & SACKS, PC;FEDERAL RESERVE PLAZA, 600 ATLANTIC AVENUE, BOSTON, MA, 02210-2211, US

(21) Appl. No.: 10/509,841

(22) PCT Filed: Apr. 9, 2003
(86) PCT No.: PCT/US03/10938

§ 371 Date: Oct. 20, 2005

Related U.S. Application Data
(60) Provisional application No. 60/371,191, filed on Apr. 9, 2002.

(51) Int. Cl. A61K 39/395

(52) U.S. Cl. 424/143.1

(57) ABSTRACT

The present invention relates to the treatment, diagnosis, and prophylactic prevention of Alzheimer's disease. More specifically, the present invention relates to methods and compositions for preventing the endocytosis and cellular internalization of integral membrane amyloid precursor protein (APP) and its subsequent catabolism by blocking or interfering with the association or binding of APP with the very low density lipoprotein receptor.

### GOVERNMENT SUPPORT

This work was funded in part by the National Institutes of Health under grant number 5R37 AG12406-06. The government may have certain rights in this invention.

### FIELD OF THE INVENTION

The present invention broadly relates to the treatment, diagnosis, and prophylactic prevention of Alzheimer's disease. More specifically, the present invention relates to methods and compositions for preventing the endocytosis and cellular internalization of integral membrane amyloid β-precursor protein (APP) and its subsequent catabolism by blocking or interfering with the association or binding of APP with the very low density lipoprotein receptor.

### BACKGROUND OF THE INVENTION

Alzheimer's disease (AD) is a late onset neurodegenerative disorder characterized by the extracellular deposition of insoluble aggregates composed of the 40 to 42 amino acid Aβ peptide in the brain (Glenner and Wong, Biochem. Biophys. Res. Commun. 120:885-890 (1984); Masters et al., EMBO J. 4:2757-2763 (1985)). Aβ peptide is derived from an integral membrane protein termed amyloid β-protein precursor protein (APP) (Tanzi et al., Science 235:880 (1987); Kang et al., Nature 325:733-736 (1987)). The function and metabolism of APP have been the subject of intensive study due to the fact that mutations in APP are associated with an autosomal dominant form of AD, (Goate et al., Nature 349:704-707 (1991)) and over-production of APP is the presumptive cause of AD in trisomy 21 (Tanzi et al., Science 235:880 (1987); Hyman et al., Proc. Natl. Acad. Sci. USA 92:3586-3590 (1995)). Multiple APP isoforms can be generated by alternatively splicing of mRNAs. The major isoforms in brain are APP695, APP751, and APP770 containing 695, 751 and 770 amino acids, respectively. These isoforms are transmembranous proteins having large extracellular regions, with hydrophobic membrane spanning domains and short cytoplasmic segments. APP is also a member of an evolutionary conserved family of proteins which include the APP-like proteins, APLP1 and APLP2 (Wasco et al., Proc. Natl. Acad. Sci. USA 89:10758-10762 (1992); Wasco et al., Nature Genet. 5:95-100 (1993); Slunt et al., J. Biol. Chem. 269:2637-2644 (1994)).

Secreted forms of APP are generated by proteolytic cleavages within their extracellular domain close to the transmembrane region. The extracellular regions of APP751, APP770, and APLP2 each contain a Kunitz protease inhibitor (KPI) domain encoded by an alternatively-transcribed exon (Kitaguchi et al., Nature 331:530-532 (1988); Tanzi et al., Nature 331:528-530 (1988); Wasco et al, Nature Genet. 5:95-100 (1993); Slunt et al., J. Biol. Chem. 269:2637-2644 (1994)). Secreted forms of APP having the KPI domain correspond to a protease inhibitor that has been identified separately and named protease nexin II (APP/PN-2) (Van Nostrand and Cunningham, J. Biol. Chem. 262:8508-8514 (1987); Oltersdorf et al., Nature 341:144-147 (1989); Van Nostrand et al., Nature 341:546-549 (1989)), a potent inhibitor of the blood coagulation factors IXa (Schmaier et al., J. Clin. Invest. 92:2540-2545 (1993)) and XIa (Van Nostrand et al., J. Biol. Chem. 265:9591-9594 (1990)). APP/PN-2 binds with high affinity to cultured fibroblasts (Johnson-Wood et al., Biochem. Biophys. Res. Commun. 200:1685-1692 (1994)), and APP/PN-2:proteinase complexes are internalized and degraded by cultured cells (Knauer and Cunningham, Proc. Natl. Acad. Sci. USA 79:2310-2314 (1982); Knauer et al., J. Cell. Physiol. 117:385-396 (1983)) although the mechanism for this process is unknown. Recent studies have identified the low density lipoprotein receptor-related protein (LRP) as the receptor responsible for the catabolism of another Kunitz-type inhibitor, tissue factor pathway inhibitor (TFPI) (Warshawsky et al., Proc. Natl. Acad. Sci. USA 91:6664-6668 (1994)).

The very low density lipoprotein (VLDL) receptor (Takahashi et al., Proc. Natl. Acad Sci. USA 89:9252-9256 (1992)) is a member of the LDL receptor family, which also includes the LDL receptor (Yamamoto et al., Cell 39:27-38 (1984)), LRP (Krieger and Herz, Annu. Rev. Biochem. 63:601-637 (1994)), and glycoprotein 330 (Saito et al., Proc. Natl. Acad. Sci. USA 91:9725-9729 (1994)). A 39 kDa protein, termed the receptor associated protein (RAP) (Strickland et al., J. Biol. Chem. 266:13364-13369 (1991)) binds to members of the LDL receptor family (Williams et al., J. Biol. Chem. 267:9035-9040 (1992); Kounnas et al., J. Biol. Chem. 267:21162-21166 (1992); Battey et al., J. Biol. Chem. 269:23268-23273 (1994)) and blocks their ligand binding capacity.

The exact mechanisms whereby APP undergoes endocytosis are not well understood. This occurs at least in part via the low-density lipoprotein receptor-related protein (LRP); LRP was recently identified as capable of binding and mediating the internalization and degradation of APP as well as its complexes with proteinases (U.S. Pat. No. 6,156,311).

After endocytosis, APP interacts with beta and gamma secretases which cleave amyloid-β from APP. Amyloid-β is then released from cells in the extracellular space, where in Alzheimer's disease it aggregates as senile plaques.

Because catabolism of APP has been shown to generate the Aβ peptide, which is believed to be the causative agent of Alzheimer's Disease, there is a need for compositions and methods which reduce the interaction, cellular internalization and subsequent catabolism of APP.

### SUMMARY OF THE INVENTION

It now has been discovered that a known endocytic receptor, VLDL-R, interacts with the amyloid precursor protein and mediates endocytosis of APP to early endosomal compartments where Aβ is formed. The APP/VLDL-R interaction can be blocked by receptor-associated protein (RAP), a 39 kilodalton chaperone protein known to block ligand receptor interactions. Accordingly, interfering with APP/VLDL-R interactions alters APP processing, and is of therapeutic value in Alzheimer's disease.

The present invention provides agents which bind to APP or VLDL-R and reduce the interaction of APP with VLDL-R, cellular internalization of APP, and subsequent catabolism of APP, as well as methods for treating Alheimer's disease using such agents to inhibit these activities of APP and/or VLDL-R.

According to one aspect of the invention, agents which bind to the APP-binding site on VLDL-R (Group I agents) and agents which bind to the VLDL-R-binding site found on APP (Group II agents) are provided. Functional derivatives or fragments of VLDL-R, RAP or APP are also provided. Preferred functional derivatives and fragments are non-hydrolyzable.

According to another aspect of the present invention, nucleic acid molecules which encode peptides and antibodies that are Group I agents and/or Group II agents and host organisms that have been transformed with the nucleic acid molecules are provided.

The present invention also provides processes for preparing nucleic acid molecules which encode a functional derivative or a fragment of VLDL-R, RAP or APP. These processes can yield nucleic acid sequences which are inserted into a vector DNA containing expression control sequences in such a way that the expression control sequences regulate the expression of the inserted nucleic acid molecule.

A further aspect of the present invention relates to methods for preparing polypeptides that are functional derivatives of VLDL-R, which include taking the polypeptide from the native receptor molecule by enzymatic, such as proteolytic, or chemical, such as reductive, treatment.

An additional aspect of the invention relates to a process for preparing Group I agents which are a functional derivative of APP or RAP which comprise expressing a recombinant nucleic acid molecule according to the invention.

The present invention further relates to Group II agents which are antibodies, or an antibody fragment containing the antigen binding domain, that bind to the VLDL-R binding site found on APP.

Another aspect of the present invention relates to processes for preparing Group II agents which are a functional derivative of VLDL-R which comprise expressing a recombinant nucleic acid molecule according to the invention.

The present invention additionally relates to hybrid cell lines that secrete monoclonal antibodies against the VLDL-receptor protein which interfere with APP attachment to the VLDL-R.

According to still another aspect of the invention, the use of Group I agents and/or Group II agents for qualitatively and/or quantitatively determining or purifying the presence of VLDL-R which is found in a sample.

A further aspect of the present invention includes a test kit for determining whether a compound, such as a polypeptide, is a Group I agent and/or a Group II agent. The kit includes a carrier means having in close confinement therein one or more container means at least one of which contains an antibody that binds to the VLDL-R binding site found on APP.

Another aspect of the present invention relates to processes for preparing antibodies that bind to the VLDL-R binding site found on APP, in which a host animal is immunized with one or more polypeptides of Group I and/or Group II, the B-lymphocytes of these host animals are fused with myeloma cells, and a hybrid cell line secreting the monoclonal antibody is subcloned and cultivated.

An additional aspect of the present invention relates to the use of Group I agents and/or Group II agents, or native VLDL-R molecules, or pharmaceutically suitable salts thereof, for the therapeutic or prophylactic treatment of the human body.

The invention also relates to methods for reducing the rate of onset or the severity of Alzheimer's disease, comprising administering to an animal, such as a human, one or more Group I agents and/or one or more Group II agents in an amount effective to reduce the rate of APP attachment to its receptor.

According to a further aspect of the present invention, pharmaceutical compositions for therapeutic treatment of Alzheimer's disease are provided. The compositions include one or more of Group I agents and/or one or more Group II agents and/or VLDL-R, and a pharmaceutically acceptable carrier.

An additional aspect of the invention relates to the use of VLDL-R for inhibiting the binding of natural ligands to a member of the LDL-receptor family of proteins.

A further aspect of the present invention relates to methods for identifying substances which inhibit the binding of a ligand (RAP) or APP to a VLDL-R polypeptide, comprising the steps of: a) incubating the VLDL receptor, or a soluble form of the receptor, with RAP or APP in the presence of a potential inhibitor substance; and b) determining the extent of binding of RAP or APP to the receptor or receptor fragment. These methods are applicable to high throughput screening of compounds to identify inhibitors of RAP or APP binding to VLDL-R.

Another aspect of the present invention relates to methods for detecting VLDL receptors, comprising the steps of: a) incubating a substance derived from a fragment of RAP or APP which contains a binding activity for the receptor with a sample; and b) determining the extent of binding of the RAP or APP material to the sample.

A further aspect of the present invention relates to methods for supplying a therapeutically active substance into a carrying cell, characterized in that a) a fragment of RAP or APP with a binding activity on the VLDL-R is coupled with the therapeutic substance; and b) the said material is added to the corresponding cell material, bound to the receptor and in this way the therapeutically active substance is introduced into the cell.

Use of the foregoing compounds and agents in the preparation of medicaments is also provided, particularly for use in treatment of Alzheimer's disease.

Other aspects, embodiments features and advantages of the present invention will be set forth in the detailed description of preferred embodiments that follows, and in part will be apparent from the description or may be learned by practice of the invention.

### BRIEF DESCRIPTION OF THE DRAWING

FIG. 1 shows the interaction of VLDL-R with APP in the absence and presence of RAP.

FIG. 2 depicts the effect of RAP on the production of Aβ peptide by CHO 13-5-1 cells transfected with human APP571.

FIG. 3 shows that anti VLDL receptor and anti-LRP IgG reduces the internalization of APP from the cell surface.

FIG. 4 depicts binding of sAPP751-Fc to LRP (FIG. 4A) or sVLDL receptor (FIG. 4B) immobilized on microtiter wells.

### DETAILED DESCRIPTION OF THE INVENTION

The current invention describes the interaction of very low density lipoprotein receptor (VLDL-R) with amyloid precursor protein (APP), as an alternate endocytic pathway for APP. It follows that interfering with endocytosis alters Aβ generation, and therefore is a therapeutic mechanism for Alzheimer's disease.

Agents that inhibit APP/VLDL-R binding and/or endocytosis are useful in accordance with the invention for reduction of Aβ production and treatment of Alzheimer's disease. These inhibitory agents can take at least two forms: (1) agents which bind to the APP-binding site on the VLDL-R protein (Group I agents) and agents which bind to the VLDL-R-binding site found on APP (Group II agents).

As used herein, an agent is said to reduce the amount or rate of binding if the amount or rate of binding is less in the presence of the agent than when the agent is absent. Under conditions when the amount or rate of reduction is nearly complete, there will be an actual inhibition or total blocking of binding.

As used herein, the agents of the present invention, i.e., the Group I agents and the Group II agents, may be any composition of matter provided that it has the ability to bind to the APP-binding site on VLDL-R (Group I) and/or the ability to bind to the VLDL-R-binding site on APP (Group II). Suitable agents exhibiting these properties include, but are not limited to, peptides, antibodies, carbohydrates, nucleic acids, vitamins, pharmaceutical agents, and the like, including derivatives thereof.

The agents of the present invention may be identified and/or prepared according to any of the methods and techniques known to those skilled in the art. These agents, particularly peptide agents and antibody agents, may occur or be produced as monomer, dimers, trimers, tetramers or multimers. Such multimers can be prepared using enzymatic or chemical treatment of the native receptor molecules or be prepared using recombinant techniques. Preferably, the agents of the present invention are selected and screened at random or rationally selected or designed using protein modeling techniques.

For random screening, candidate agents are selected at random and assayed for their ability to reduce the amount or rate of binding of the amyloid β-precursor protein (APP) to the VLDL receptor. Any of the suitable methods and techniques known to those skilled in the art may be employed to assay candidate agents.

For rational selection or design, the agent is selected based on the configuration of the VLDL-R binding site found on APP or the APP binding site found on the VLDL-R. Any of the suitable methods and techniques known to those skilled in the art may be employed for rational selection or design. For example, one skilled in the art can readily adapt currently available procedures to generate antibodies, peptides, pharmaceutical agents and the like capable of binding to a specific peptide sequence of VLDL-R or APP. Illustrative examples of such available procedures are described, for example, in Hurby et al., “Application of Synthetic Peptides: Antisense Peptides,” in Synthetic Peptides, A User's Guide, W. H. Freeman, N.Y., pp. 289-307 (1992); Kaspczak et al., Biochemistry 28:9230 (1989); and Harlow, Antibodies, Cold Spring Harbor Press, N.Y. (1990).

The agents of the present invention can alternatively be identified using modification of methods known in the art. For example, suitable peptide agents may be identified using the filter binding assay described by Mischak et al. (Mischak et al., J. Gen. Virol. 69:2653-2656 (1988) and Mischak et al., Virology 163:19-25 (1988)), wherein the peptide is applied to a suitable membrane, such as nitrocellulose, and the membrane is saturated with a detergent mixture in order to block any non-specific binding. The treated membrane is then incubated with labeled rhinovirus, e.g. with HRV2 labeled with ³⁵S-methionine, in order to check the specific binding. After washing and drying of the membrane, specific binding can be visualized by autoradiography.

Other methods include fluorescence energy transfer assays, as described in the Examples. Briefly, one can identify inhibitors of APP/VLDL-R interaction by observing differences in fluorescence energy transfer of the fluorescently labeled VLDL-R in the presence and absence of potential agents.

As noted above, the Group I agents of the present invention include those agents which bind directly to the APP binding site found on the VLDL-R. Additionally, the Group I agents of the present invention bind to the VLDL-R in interfering proximity with the APP binding site or bind to the VLDL-R in such a manner so as to conformationally alter the APP binding site. Suitable Group I agents can therefore be first identified by their ability to bind the VLDL-R and then by their ability to reduce the amount or rate at which APP binds to VLDL-R. Illustrative examples of Group I agents of the present invention include, but are not limited to: soluble fragments of APP containing the KPI domain; anti-VLDL-R antibodies or binding fragments thereof; soluble fragments of receptor associated protein (RAP); α₂-macroglobulin:proteinase complexes; pregnancy zone protein (PZP):proteinase complexes; tissue-type plasminogen activator; pro-urokinase-type plasminogen activator; tissue factor pathway inhibitor; apolipoprotein E-enriched lipoproteins; lipoprotein lipase; hepatic lipase; thrombospondin; and lactoferrin.

Preferred Group I agents are based on and derived from the amino acid sequence of the receptor associated protein (RAP). An especially preferred type of Group I agent is isolated RAP or a fragment thereof, such as a soluble fragment of RAP which contains the VLDL-R binding site, particularly one which binds selectively (i.e., specifically) to VLDL-R and not to other members of the LDL receptor protein family. Such agents act as competitive inhibitors of APP binding to its receptor in vitro as well as in vivo.

The preferred fragments of RAP are soluble under physiological conditions. The C-terminus of these polypeptides can be shortened as desired, provided that the binding capacity for the VLDL-R protein remains intact. The preferred amino acid sequence of RAP corresponds to the human protein. Suitable RAP sequences can also be derived from the amino acid sequence of RAP isolated from other mammals or amphibians.

RAP, or a fragment thereof, may be produced using any of the methods and techniques known to those skilled in the art. For example, RAP can be purified from a source which naturally expresses the protein, can be isolated from a recombinant host which has been altered to express RAP or fragment thereof, or can be synthesized using protein synthesis techniques known in the art. The skilled artisan can readily adapt a variety of techniques in order to obtain Group I peptide agents which contain the VLDL-R binding site found on RAP.

The isolation of native RAP proteins is known, as described, for example, in Ashcom et al., J. Cell. Biol. 110:1041-1048 (1990) and Jensen et al., FEBS Lett. 255:275-280 (1989). In order to generate fragments of RAP which contains the VLDL-R binding site, isolated native protein may be converted by enzymatic and/or chemical cleavage to generate fragments of the whole protein, for example by reacting cell lines which express an RAP with an enzyme such as papain or trypsin or a chemical such as cyanogen bromide. Proteolytically active enzymes or chemicals are preferably selected in order to release the extracellular receptor region. Fragments which contain the VLDL-R binding site, especially fragments which are soluble under physiological conditions, can then be isolated using known methods.

Alternatively, RAP or a fragment of RAP may be expressed in a recombinant bacteria, as described, for example, in Williams et al., J. Biol. Chem. 267:9035-9040 (1992) and Wurshawsky et al., J. Biol. Chem. 269:3325-3330 (1994).

The Group II agents of the present invention include compositions which bind to the VLDL-R binding site found on APP. Additionally, the Group II agents of the present invention include compositions that bind to APP in interfering proximity to the VLDL-R binding site. Suitable Group II agents can therefore be first identified by their ability to bind to APP and then by their ability to reduce the amount or rate at which APP binds to VLDL-R. Illustrative examples of the Group II agents of the present invention include, but are not limited to, antibodies which bind to the VLDL-R binding site found on APP and soluble fragments of VLDL-R.

Preferred Group II agents include antibodies and antibody fragments which are capable of binding to a residue found on APP and consequently act as a competitive inhibitor for VLDL-R binding. The most preferred antibodies or antibody fragments of the present invention bind to an VLDL-R-specific epitope in APP. Antibodies, or other Group II agents such as anti-sense peptides, which bind to epitopes within this sequence reduce the amount or rate of APP binding to VLDL-R.

The antibodies of the present invention include polyclonal and monoclonal antibodies, as well as antibody fragments and derivatives that contain the relevant antigen binding domain of the antibodies. Such antibodies or antibody fragments are preferably used in the diagnostic and therapeutic embodiments of the present invention.

Suitable monoclonal and polyclonal antibodies may be prepared by any of the methods and techniques well known in the art, such as described in, for example, A. M. Campbell, Monoclonal Antibody Technology: Laboratory Techniques in Biochemistry and Molecular Biology, Elsevier Science Publishers, Amsterdam, The Netherlands (1984) and Harlow; Antibodies, Cold Spring Harbor Press, N.Y. (1989). For example, an antibody capable of binding to a domain of APP can be generated by immunizing an animal with a polypeptide whose sequence is encoded by that domain. Any animal (mouse, rabbit, etc.) which is known to produce antibodies can be utilized to produce antibodies with the desired specificity and suitable methods for immunization of these animals are well known in the art, including, for example, subcutaneous or intraperitoneal injection of the polypeptide. One skilled in the art will recognize that the amount of polypeptide used for immunization will vary based on a number of factors, including the animal which is immunized, the antigenicity of the polypeptide selected, and the site of injection.

The polypeptides used as an immunogen may be modified as appropriate or administered in an adjuvant in order to increase the peptide antigenicity. Suitable methods increasing antigenicity are well known in the art, and include, for example, coupling the antigen with a heterologous protein (such as globulin or β-galactosidase) or through the inclusion of an adjuvant during immunization.

A preferred method of generating monoclonal antibodies comprises removing spleen cells from the immunized animals, fusing these cells with myeloma cells, such as SP2/0-Ag14 myeloma cells, and allowing them to become monoclonal antibody-producing hybridoma cells. Any one of a number of methods well known in the art may be used to identify the hybridoma cell which produces an antibody with the desired characteristics. These include screening the hybridomas with an ELISA assay, western blot analysis, or radioimmunoassay (Lutz et al., Exp. Cell Res. 175:109-124 (1988); Kishimoto et al., Proc. Natl. Acad. Sci USA 87:2244-2248 (1990)). Hybridomas secreting the desired antibodies are cloned and the class and subclass of the secreted antibodies are determined using procedures known in the art (Campbell, A. M., Monoclonal Antibody Technology: Laboratory Techniques in Biochemistry and Molecular Biology, Elsevier Science Publishers, Amsterdam, The Netherlands (1984)).

For polyclonal antibodies, antibody-containing antisera is preferably isolated from the immunized animal and is screened for the presence of antibodies with the desired specificity using one of the above-described procedures.

The present invention further provides hybrid cell lines which secrete monoclonal antibodies selective for the Group I agents and/or Group II agents. These monoclonal antibodies are capable of wholly or partially neutralizing the activity of the polypeptides or specifically binding to one of the said polypeptides. These monoclonal antibodies can be used for qualitative and/or quantitative measurement or for purification of the polypeptides according to the invention. The present invention therefore also includes test systems which contain the monoclonal antibodies herein described.

Antibodies may be used as an isolated whole antibody, or can be used as a source for generating antibody fragments which contain the antigen binding site of the antibody. Examples of such antibody fragments include, but are not limited to the F, the F(ab), the F(ab)₂, fragment, as well as single chain antibodies.

Significantly, as is well-known in the art, only a small portion of an antibody molecule, the paratope, is involved in the binding of the antibody to its epitope (see, in general, Clark, W. R. (1986) The Experimental Foundations of Modem Immunology Wiley & Sons, Inc., New York; Roitt, I. (1991) Essential Immunology, 7th Ed., Blackwell Scientific Publications, Oxford). The pFc′ and Fc regions, for example, are effectors of the complement cascade but are not involved in antigen binding. An antibody from which the pFc′ region has been enzymatically cleaved, or which has been produced without the pFc′ region, designated an F(ab′)₂ fragment, retains both of the antigen binding sites of an intact antibody. Similarly, an antibody from which the Fc region has been enzymatically cleaved, or which has been produced without the Fc region, designated an Fab fragment, retains one of the antigen binding sites of an intact antibody molecule. Proceeding further, Fab fragments consist of a covalently bound antibody light chain and a portion of the antibody heavy chain denoted Fd. The Fd fragments are the major determinant of antibody specificity (a single Fd fragment may be associated with up to ten different light chains without altering antibody specificity) and Fd fragments retain epitope-binding ability in isolation.

Within the antigen-binding portion of an antibody, as is well-known in the art, there are complementarity determining regions (CDRs), which directly interact with the epitope of the antigen, and framework regions (FRs), which maintain the tertiary structure of the paratope (see, in general, Clark, 1986; Roitt, 1991). In both the heavy chain Fd fragment and the light chain of IgG immunoglobulins, there are four framework regions (FR1 through FR4) separated respectively by three complementarity determining regions (CDR1 through CDR3). The CDRs, and in particular the CDR3 regions, and more particularly the heavy chain CDR3, are largely responsible for antibody specificity.

It is now well-established in the art that the non-CDR regions of a mammalian antibody may be replaced with similar regions of conspecific or heterospecific antibodies while retaining the epitopic specificity of the original antibody. This is most clearly manifested in the development and use of “humanized” antibodies in which non-human CDRs are covalently joined to human FR and/or Fc/pFc′ regions to produce a functional antibody.

Various methods known in the art can be used to generate such fragments without undue experimentation. Recombinant techniques are preferred for generating large quantities of antibodies, antibody fragments and single chain antibodies, as described, for example, in Pluckthum, Bio/Technology 10:163-167 (1992); Carter et al., Bio/Technology 10:167-170 (1992); and Mullinax et al., Biotechniques 12:864-869 (1992). In addition, recombinant techniques may be used to generate heterobifunctional antibodies.

In general, recombinant production of antibodies, antibody fragments or derivatives thereof, uses mRNA encoding an antibody which is isolated from hybridoma cells that produce the desired antibody. This mRNA is then used as a source for generating a cDNA molecule which encodes the antibody, or a fragment thereof. Once obtained, the cDNA may be amplified and expressed according to known methods in a variety of eukaryotic and prokaryotic hosts.

The present invention further includes derivatives of antibodies (antibody derivatives). As used herein, an “antibody derivatives” contain an antibody of the present invention, or a fragment thereof, as well as an additional moiety which is not normally a part of the antibody. Such moieties may improve the solubility, absorption, biological half-life, etc., of the antibody, decrease the toxicity of the antibody, eliminate or attenuate any undesirable side effect of the antibody, or serve as a detectable marker of the presence of the antibody. Moieties capable of mediating such effects are well known in the art.

Detectably labeled antibodies constitute a special class of the antibody derivatives of the present invention. An antibody is said to be “detectably labeled” if the antibody, or fragment thereof, is attached to a molecule which is capable of identification, visualization, or localization using known methods. Suitable detectable labels include radioisotopic labels, enzyme labels, non-radioactive isotopic labels, fluorescent labels, toxin labels, affinity labels, chemiluminescent labels and nuclear magnetic resonance contrast agents.

Illustrative examples of suitable enzyme labels include, but are not limited to, luciferase, malate dehydrogenase, staphylococcal nuclease, delta-5-steroid isomerase, yeast-alcohol dehydrogenase, alpha-glycerol phosphate dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase, and acetylcholine esterase.

Examples of suitable radioisotopic labels include, but are not limited to, ³H, ¹¹¹In, ¹²⁵I, ¹³¹I, ³²P, ³⁵S, ¹⁴C, ⁵¹Cr, ⁵⁷To, ⁵⁸Co, ⁵⁹Fe, ⁷⁵Se, ¹⁵²Eu, ⁹⁰Y, ⁶⁷Cu, ²¹⁷Ci, ²¹¹At, ²¹²Pb, ⁴⁷Sc, ¹⁰⁹Pd, etc. ¹¹¹In is a preferred isotope where in vivo imaging is used since its avoids the problem of dehalogenation of the ¹²⁵I or ¹³¹I-labeled monoclonal antibody by the liver. In addition, this radionucleotide has a more favorable gamma emission energy for imaging (Perkins et al., Eur. J. Nucl. Med 10:296-301 (1985); Carasquillo et al., J. Nucl. Med. 28:281-287 (1987)). For example, ¹¹¹In coupled to monoclonal antibodies with 1-(p-isothiocyanatobenzyl)-DPTA has shown little uptake in non-tumorous tissues, particularly the liver, and therefore enhances specificity of tumor localization (Esteban et al., J. Nucl. Med. 28:861-870 (1987)).

Illustrative examples of suitable non-radioactive isotopic labels include, but are not limited to, ¹⁵⁷Gd, ⁵⁵Mn, ¹⁶²Dy, ⁵²Tr, and ⁵⁶Fe.

Illustrative examples of suitable fluorescent labels include, but are not limited to, an ¹⁵²Eu label, a fluorescent protein (including green fluorescent protein (GFP), enhanced green fluorescent protein (EGFP) and yellow fluorescent protein (YFP)), a fluorescein label, an isothiocyanate label, a rhodamine label, a phycoerythrin label, a phycocyanin label, an allophycocyanin label, an o-phthaldehyde label, and a fluorescamine label.

Suitable chemical toxins or chemotherapeutic agents include members of the enediyne family of molecules, such as calicheamicin and esperamicin. Chemical toxins can also be taken from the group consisting of duocarmycin (see e.g., U.S. Pat. No. 5,703,080 and U.S. Pat. No. 4,923,990), methotrexate, doxorubicin, melphalan, chlorambucil, ARA-C, vindesine, mitomycin C, cis-platinum, etoposide, bleomycin and 5-fluorouracil. Toxins that are less preferred in the compositions and methods of the invention include poisonous lectins, plant toxins such as ricin, abrin, modeccin, botulinum and diphtheria toxins. Of course, combinations of the various toxins could also be coupled to one antibody molecule thereby accommodating variable cytotoxicity. Other chemotherapeutic agents are known to those skilled in the art.

Illustrative examples of chemiluminescent labels include a luminal label, an isoluminal label, an aromatic acridinium ester label, an imidazole label, an acridinium salt label, an oxalate ester label, a luciferin label, a luciferase label, and an aequorin label.

Illustrative examples of nuclear magnetic resonance contrasting agents include paramagnetic heavy metal nuclei such as Gd, Mn, and Fe.

The coupling of one or more molecules to antibodies is envisioned to include many chemical mechanisms, for instance covalent binding, affinity binding, intercalation, coordinate binding, and complexation

The covalent binding can be achieved either by direct condensation of existing side chains or by the incorporation of external bridging molecules. Many bivalent or polyvalent agents are useful in coupling protein molecules to other proteins, peptides or amine functions, etc. For example, the literature is replete with coupling agents such as carbodiimides, diisocyanates, glutaraldehyde, diazobenzenes, and hexamethylene diamines. This list is not intended to be exhaustive of the various coupling agents known in the art but, rather, is exemplary of the more common coupling agents.

In preferred embodiments, it is contemplated that one may wish to first derivatize the antibody, and then attach the toxin component to the derivatized product. Suitable cross-linking agents for use in this manner include, for example, SPDP (N-succinimidyl-3-(2-pyridyldithio)propionate), and SMPT, 4-succinimidyl-oxycarbonyl-α-methyl-α (2-pyridyldithio)toluene.

Radionuclides typically are coupled to an antibody by chelation. For example, in the case of metallic radionuclides, a bifunctional chelator is commonly used to link the isotope to the antibody or other protein of interest. Typically, the chelator is first attached to the antibody, and the chelator-antibody conjugate is contacted with the metallic radioisotope. A number of bifunctional chelators have been developed for this purpose, including the dithylenetriamine pentaacetic acid (DTPA) series of amino acids described in U.S. Pat. Nos. 5,124,471, 5,286,850 and 5,434,287, which are incorporated herein by reference. As another example, hydroxamic acid-based bifunctional chelating agents are described in U.S. Pat. No. 5,756,825, the contents of which are incorporated herein. Another example is the chelating agent termed p-SCN-Bz-HEHA (1,4,7,10,13,16-hexaazacyclo-octadecane-N,N′,N″,N′″,N″″,N′″″-hexaacetic acid) (Deal et al., J. Med. Chem. 42:2988, 1999), which is an effective chelator of radiometals such as ²²⁵Ac.

In yet other embodiments, the antibodies can be chimeric or humanized antibodies. As used herein, the term “chimeric antibody” refers to an antibody, that combines the murine variable or hypervariable regions with the human constant region or constant and variable framework regions. As used herein, the term “humanized antibody” refers to an antibody that retains only the antigen-binding CDRs from the parent antibody in association with human framework regions (see, Waldmann, 1991, Science 252:1657). Such chimeric or humanized antibodies retaining binding specificity of the murine antibody are expected to have reduced immunogenicity when administered in vivo for diagnostic, prophylactic or therapeutic applications according to the invention.

In certain embodiments, the antibodies are human antibodies. The term “human antibody”, as used herein, is intended to include antibodies having variable and constant regions derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse have been grafted onto human framework sequences (referred to herein as “humanized antibodies”). Human antibodies directed against PSMA can be generated using transgenic mice carrying parts of the human immune system rather than the mouse system.

Fully human monoclonal antibodies also can be prepared by immunizing mice transgenic for large portions of human immunoglobulin heavy and light chain loci. See, e.g., U.S. Pat. Nos. 5,591,669, 5,598,369, 5,545,806, 5,545,807, 6,150,584, and references cited therein, the contents of which are incorporated herein by reference. These animals have been genetically modified such that there is a functional deletion in the production of endogenous (e.g., murine) antibodies. The animals are further modified to contain all or a portion of the human germ-line immunoglobulin gene locus such that immunization of these animals will result in the production of fully human antibodies to the antigen of interest. Following immunization of these mice (e.g., XenoMouse (Abgenix), HuMAb mice (Medarex/GenPharm)), monoclonal antibodies can be prepared according to standard hybridoma technology. These monoclonal antibodies will have human immunoglobulin amino acid sequences and therefore will not provoke human anti-mouse antibody (HAMA) responses when administered to humans.

Another type of the Group II agents of the present invention are peptide agents which are classified as antisense-peptide sequences. Antisense-peptide sequences are short peptides which are specifically designed to bind to a particular amino acid sequence. In general, such antisense peptide agents may be generated using methods known in the art, such as those described, for example, in Hurby et al., “Application of Synthetic Peptides: Antisense Peptides,” in Synthetic Peptides, A User's Guide, W. H. Freeman, N.Y., pp. 289-307 (1992) and Kaspczak et al., Biochemistry 28:9230-8 (1989).

An additional class of the Group II agents of the invention are natural ligands of APP. As used herein, a natural ligand of APP is defined as any substance which binds to APP, such as soluble fragments of VLDL-R containing the APP binding site. Such soluble fragments may be prepared by any suitable method known to those skilled in the art, such as the method of Davis et al., Nature 326:760-765 (1987), which involves deletion of the entire EGF domain. Moreover, soluble forms of the receptor may be formed by inserting a stop codon in front of the region of DNA encoding the cytoplasmic or transmembrane domain (Yokade et al., J. Cell. Biol. 117:39 (1992)).

The agents of the present invention may be used in vitro and/or in vivo to study VLDL-R attachment and to reduce the rate of onset and/or ameliorate the duration and severity of Alzheimer's disease. In addition, the agents of the present invention may be used in qualitative, quantitative and preparative assays and purification procedures to isolate, identify and facilitate the purification of APP.

For in vivo use, the agents of the present invention may be provided to a patient as a means of reducing the amount or rate of APP binding to VLDL-R (Hayden, et al., Antiviral Res. 9:233-247 (1988)).

The present invention therefore provides pharmaceutical compositions comprising a Group I agent and/or a Group II agent. These pharmaceutical compositions may be administered orally, rectally, parenterally, intracistemally, intravaginally, intraperitoneally, topically (as by powders, ointments, drops or transdermal patch), bucally, or as an oral or nasal spray. As used herein, “pharmaceutically acceptable carrier” is intended to mean a non-toxic solid, semisolid or liquid filler, diluent, encapsulating material or formulation auxiliary of any type. The term “parenteral” as used herein refers to modes of administration which include intravenous, intramuscular, intraperitoneal, intrasternal, subcutaneous and intraarticular injection and infusion. One of ordinary skill will recognize that the choice of a particular mode of administration can be made empirically based upon considerations such as the particular disease state being treated; the type and degree of the response to be achieved; the specific agent or composition employed; the age, body weight, general health, sex and diet of the patient; the time of administration and rate of excretion of the agent or composition; the duration of the treatment; drugs (such as a chemotherapeutic agent) used in combination or coincidental with the specific composition; and like factors well known in the medical arts.

Pharmaceutical compositions of the present invention for parenteral injection may comprise pharmaceutically acceptable sterile aqueous or nonaqueous solutions, dispersions, suspensions or emulsions as well as sterile powders for reconstitution into sterile injectable solutions or dispersions just prior to use. Illustrative examples of suitable aqueous and nonaqueous carriers, diluents, solvents or vehicles include, but are not limited to, water, ethanol, polyols (such as glycerol, propylene glycol, polyethylene glycol, and the like), carboxymethylceuulose and suitable mixtures thereof, vegetable oils (such as olive oil), and injectable organic esters such as ethyl oleate. Proper fluidity can be maintained, for example, by the use of coating materials such as lecithin, by the maintenance of the required particle size in the case of dispersions, and by the use of surfactants.

The compositions of the present invention may also contain adjuvants such as preservatives, wetting agents, emulsifying agents, and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents such as sugars, sodium chloride, and the like. Prolonged absorption of the injectable pharmaceutical form may be brought about by the inclusion of agents which delay absorption such as aluminum monostearate and gelatin.

In some cases, in order to prolong the effect of the therapeutic agent or inhibitor, it is desirable to slow the absorption from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissues.

The injectable formulations can be sterilized, for example, by filtration through a bacterial-retaining filter, or by incorporating sterilizing agents in the form of sterile solid compositions which can be dissolved or dispersed in sterile water or other sterile injectable medium just prior to use.

Solid dosage forms for oral administration include, but are not limited to, capsules, tablets, pills, powders, and granules. In such solid dosage forms, the active compounds are preferably mixed with at least one pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents as appropriate.

Solid compositions of a similar type may also be employed as fillers in soft and hard filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Illustrative examples of embedding compositions which can be used include polymeric substances and waxes.

The active agents of Group I and/or Group II can also be in micro-encapsulated form, if appropriate, with one or more of the above-mentioned excipients.

Liquid dosage forms for oral administration include, but are not limited to, pharmaceutically acceptable emulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

Besides inert diluents, the oral compositions may also contain adjuvants such as wetting agents, emulsifying and suspending agents, sweetening, flavoring, and perfuming agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar, and tragacanth, and mixtures thereof

The agent or inhibitor can also be administered in the form of liposomes. As is known to those skilled in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes can be used. The present compositions in liposome form can contain, in addition to the agent or inhibitor, stabilizers, preservatives, excipients, and the like. Preferred lipids are phospholipids and phosphatidyl cholines (lecithins), both natural and synthetic. Methods to form liposomes are known in the art. See, e.g., Prescott, ed., METHODS IN CELL BIOLOGY, Volume XIV, Academic Press, New York, N.Y. (1976), p. 33 et seq.

The agents of the present invention can be formulated according to known methods to prepare pharmaceutically acceptable compositions, whereby these materials, or their functional derivatives, are combined in a mixture with a pharmaceutically acceptable carrier vehicle. Suitable vehicles and their formulation, inclusive of other human proteins, e.g., human serum albumin, are well known in the art. In order to form a pharmaceutically acceptable composition suitable for effective administration, such compositions will contain an effective amount of one or more agents of the present invention.

Additional pharmaceutical methods may be employed to control the duration of action. Controlled release preparations may be achieved through the use of polymers to complex or absorb the therapeutic agents of the invention. The controlled delivery may be exercised by selecting appropriate macromolecules (such as polyesters, polyamino acids, polyvinyl, pyrrolidone, ethylenevinylacetate, methylcellulose, carboxymethylcellulose, or protamine sulfate) and methods of incorporation in order to control release. Another possible method to control the duration of action by controlled release preparations is to incorporate antibodies into particles of a polymeric material such as polyesters, polyamino acids, hydrogels, poly(lactic acid) or ethylene vinyl acetate copolymers. Alternatively, instead of incorporating these agents into polymeric particles, it is possible to entrap these materials in microcapsules prepared, for example, by coacervation techniques or by interfacial polymerization, for example, hydroxymethylcellulose or gelatine-microcapsules and poly(methylmethacylate) microcapsules, respectively, or in colloidal drug delivery systems, for example, liposomes, albumin microspheres, microemulsions, nanoparticles, and nanocapsules or in macroemulsions.

The pharmaceutical formulations of the present invention are prepared, for example, by admixing the active agent with solvents and/or carriers, optionally using emulsifiers and/or dispersants, whilst if water is used as the diluent, organic solvents may be used as solubilizing agents or auxiliary solvents. As described above, the excipients used include, for example, water, pharmaceutically acceptable organic solvents such as paraffins, vegetable oils, mono- or polyfunctional alcohols, carriers such as natural mineral powders, synthetic mineral powders, sugars, emulsifiers and lubricants.

One of ordinary skill will appreciate that effective amounts of the inventive therapeutic agents can be determined empirically and may be employed in pure form or, where such forms exist, in pharmaceutically acceptable salt, ester or prodrug form. The agonist or antagonist my be administered in compositions in combination with one or more pharmaceutically acceptable excipients. It will be understood that, when administered to a human patient, the total daily usage of the agents and compositions of the present invention will be decided by the attending physician within the scope of sound medical judgement. The specific therapeutically effective dose level for any particular patient will depend upon a variety of factors including the type and degree of the response to be achieved; the specific agent or composition employed; the age, body weight, general health, sex and diet of the patient; the time of administration, route of administration, and rate of excretion of the agent or composition; the duration of the treatment; drugs (such as a chemotherapeutic agent) used in combination or coincidental with the specific composition; and like factors well known in the medical arts.

Techniques of dosage determination are well known in the art for antibody and peptide agents. In general, it is desirable to provide a patient with a dosage of antibody or peptide agent in the range of from about 1 pg/kg to 10 mg/kg (body weight of patient). The therapeutically effective dose can be lowered if the agent of the present invention is additionally administered with another compound. As used herein, one compound is said to be additionally administered with a second compound when the administration of the two compounds is in such proximity of time that both compounds can be detected at the same time in the patient's serum.

For example, satisfactory results are obtained by oral administration of therapeutic dosages on the order of from 0.05 to 10 mg/kg/day, preferably 0.1 to 7.5 mg/kg/day, more preferably 0.1 to 2 mg/kg/day, administered once or, in divided doses, 2 to 4 times per day. On administration parenterally, for example by i.v. drip or infusion, dosages on the order of from 0.01 to 5 mg/kg/day, preferably 0.05 to 1.0 mg/kg/day and more preferably 0.1 to 1.0 mg/kg/day can be used. Suitable daily dosages for patients are thus on the order of from 2.5 to 500 mg p.o., preferably 5 to 250 mg p.o., more preferably 5 to 100 mg p.o., or on the order of from 0.5 to 250 mg i.v., preferably 2.5 to 125 mg i.v. and more preferably 2.5 to 50 mg i.v.

Dosaging may also be arranged in a patient specific manner to provide a predetermined concentration of an agent in the blood, as determined by the RIA technique. Thus patient dosaging may be adjusted to achieve regular on-going trough blood levels, as measured by RIA, on the order of from 50 to 1000 ng/ml, preferably 150 to 500 ng/ml.

The agents of the present invention are intended to be provided to a patient in an amount sufficient to reduce the amount or rate of binding of human APP to VLDL-R, i.e., and effective amount. An amount is said to be sufficient to “reduce the amount or rate of APP binding” if the dosage, route of administration, etc. of the agent is sufficient to reduce the amount or rate of APP attachment to the VLDL receptor. Such an effect can be assayed, for example, by examining the onset of Alzheimer's disease symptoms occurring in vivo, or by correlating in vitro blocking studies with predicted in vivo efficacy.

The administration of the agents of the present invention may be for either prophylactic or therapeutic purpose. When provided prophylactically, the agent is provided in advance of any Alzheimer's disease symptoms. The prophylactic administration of the agent serves to prevent or reduce the rate of onset of symptoms. When provided therapeutically, the agent is provided at (or shortly after) the onset of the appearance of symptoms of actual disease. The therapeutic administration of the agent serves to reduce the severity and duration of Alzheimer's disease.

The present invention further includes the use of the agents of the present invention in diagnostic applications. The Group I agents of the present invention can be used to detect the presence of VLDL-R in a test sample. The Group II agents of the present invention can be used to detect the presence of APP in a test sample.

Conditions for incubating an agent with a test sample vary. Incubation conditions will depend on factors such as the type of agent, format, and detection system employed for the assay, as well as the nature of the test sample used in the assay. For example, condition will vary slightly when a whole antibody, a single chain antibody, a F(ab) fragment, or a peptide agent is used. One skilled in the art will recognize that any one of the commonly available immunological assay formats (such as radioimmunoassays, enzyme-linked immunosorbent assays, diffusion based ouchterlony, or rocket immunofluorescent assays) can readily be adapted to employ the antibodies of the present invention. Examples of such assays can be found in T. Chard, An Introduction to Radioimmunoassay and Related Techniques, Elsevier Science Publishers, Amsterdam, The Netherlands (1986); G. R. Bullock et al., Techniques in Immunocytochemistry, Academic Press, Orlando, Fla. Vol. 1 (1982), Vol. 2 (1983), Vol. 3 (1985); and P. Tijssen, P., Practice and Theory of Enzyme Immunoassays: Laboratory Techniques in Biochemistry and Molecular Biology, Elsevier Science Publishers, Amsterdam, The Netherlands (1985).

In one embodiment of the above-described method, the agent of the present invention is immobilized on a solid support for use in the diagnostic assay. Illustrative examples of such solid supports include, but are not limited to, plastics such as polycarbonate, complex carbohydrates such as agarose and sepharose, and acrylic resins, such as polyacrylamide and latex beads. Techniques for coupling agents such as antibodies, peptides and the like to such solid supports are well known in the art, as described, for example, in D. M. Weir et al., Handbook of Experimental Immunology, 4th Ed., Blackwell Scientific Publications, Oxford, England, Chapter 10 (1986) and W. D. Jacoby et al., Meth. Enzym. 34, Academic Press, N.Y. (1974).

Additionally, one or more of the agents of the present invention which is used in one of the above-described methods can be detectably labeled prior to use, for example, through the use of radioisotopes, affinity labels (such as biotin, avidin, etc.), enzymatic labels (such as horse radish peroxidase, alkaline phosphatase, etc.) fluorescent labels (such as FITC or rhodamine, etc.), paramagnetic atoms, etc. Procedures for accomplishing such labeling are well-known in the art (L. A. Sternberger et al., J. Histochem. Cytochem. 18:315 (1970); E. A. Bayer et al., Meth. Enzym. 62:308 (1979); E. Engval et al., Immunol. 109:129 (1972); and J. W. Goding, J. Immunol. Meth. 13:215 (1976)).

The materials used in the inventive assays are ideally suited for the preparation of a kit. For example, the present invention provides a compartmentalized kit to receive in close confinement, one or more containers which comprises: a) a first container comprising an agent capable of binding to the VLDL-R binding site; and b) one or more other containers comprising one or more of the following: wash reagents and reagents capable of detecting the presence of bound agents from the first container.

As used herein, a compartmentalized kit includes any kit in which reagents are contained in separate containers. Illustrative examples of such containers include, but are not limited to, small glass containers, plastic containers or strips of plastic or paper. Particularly preferred types of containers allow the skilled worker to efficiently transfer reagents from one compartment to another compartment such that the samples and reagents are not cross-contaminated and the agents or solutions of each container can be added in a quantitative fashion from one compartment to another. Such containers include, but are not be limited to, a container which will accept the test sample, a container which contains one or more of the agents of the present invention used in the assay, containers which contain wash reagents (such as phosphate buffered saline, Tris-buffers, etc.), and containers which contain the reagents used to detect the bound agent.

The types of detection reagents which can be used in the above described kits include, but are not limited to, labeled secondary agents, or in the alternative, if the primary agent is labeled, enzymatic or agent binding reagents which are capable of reacting with the labeled agent. One skilled in the art will readily recognize that the agents of the present invention can be readily incorporated into one of the established kit formats which are well known in the art.

A further aspect of the present invention concerns DNA molecules which encode for the polypeptide and antibody agents of Group I and/or Group II. The starting nucleotide molecules can be obtained by the person skilled in the art using known methods. Moreover, the DNA molecules, where the amino acid sequence is known, may be produced synthetically or by amplification methods such as PCR (Sambrook et al., Molecular Cloning: A Laboratory Manual, Cold Spring Harbor Laboratory Press (1989)). The DNA sequences of the present invention include not only the actual nucleotide sequence used by the organism from which the receptor protein is derived but also includes all degenerate forms which encode a peptide with the desired sequence.

The invention includes DNA sequences which have been modified utilizing methods known in the art, such as those generated by mutation, deletion, transposition or addition. The preferred mutations will introduce stop codons within the RAP sequence so that a truncated protein will be generated.

The present invention further includes DNA vectors which contain the DNA sequences described above and below. In particular, these may be vectors in which the DNA molecules described are functionally linked to control sequences which allows expression of the corresponding polypeptides. These are preferably plasmids which can be replicated and/or expressed in prokaryotes such as E. coli and/or in eukaryotic systems such as yeasts or mammalian cell lines. These vectors may also be mammalian viral vectors which can be replicated and/or expressed in eukaryotes such as mammalian cell lines and in the human patient, as “host,” for integration into the cellular genome of the patient and expression as genetic therapy systems.

The invention also includes host organisms transformed with the above vectors. Expression in prokaryotes and eukaryotes may be carried out using techniques known in the art. The DNA sequences according to the invention may be expressed as fusion polypeptides or as intact, native polypeptides. Fusion proteins may advantageously be produced in large quantities. They are generally more stable than the native polypeptide and are easy to purify. The expression of these fusion proteins can be controlled by normal host DNA sequences.

For example, the DNA sequences according to the invention can be cloned and expressed as lacZ fusion genes in E. coli. A person skilled in the art has a variety of vector systems available for this purpose, e.g. the pUR-vector series (U. Ruther and B. Muller-Hill, EMBO J. 2:1791 (1983)). The bacteriophage promoter λp_{R} may also be used, in the form of the vectors pEX-1 to -3, for expressing large amounts of Cro-β-galactosidase fusion protein (K. K. Stanley and J. P. Luzio, EMBO J. 3:1429 (1984)). Analogously, the tac promoter which can be induced with IPTG can also be used, for example in the form of the pROK-vector series (CLONTECH Laboratories).

The prerequisite for producing intact native polypeptides using E. coli is the use of a strong, regulatable promoter and an effective ribosome binding site. Promoters which may be used for this purpose include the temperature sensitive bacteriophage λp_{L}-promoter, the tac-promoter inducible with IPTG or the T7-promoter. Numerous plasmids with suitable promoter structures and efficient ribosome binding sites have been described, such as for example pKC30 (λp_{L}; Shimatake and Rosenberg, Nature 292:128 (1981), pKK173-3 (tac, Amann and Brosius, Gene 40:183 (1985)) or pET-3 (T7-promoter (Studier and Moffat, J. Mol. Biol. 189:113 (1986)).

A number of other suitable vector systems for expressing the DNA according to the invention in E. coli are known from the prior art and are described, for example, in Sambrook et al., Molecular Cloning: A Laboratory Manual, Cold Spring Harbor Laboratory Press (1989)).

Suitable E. coli strains which are specifically tailored to a particular expression vector are known to those skilled in the art (Sambrook et al., Molecular Cloning: A Laboratory Manual, Cold Spring Harbor Laboratory Press (1989)). The experimental performance of the cloning experiments, the expression of the polypeptides in E. coli and the working up and purification of the polypeptides are known and are described, for example, in Sambrook et al., Molecular Cloning: A Laboratory Manual, Cold Spring Harbor Laboratory Press (1989). In addition to prokaryotes, eukaryotic microorganisms such as yeast may also be used.

For expression in yeast, the plasmid YRp7 (Stinchcomb et al. Nature 282:39 (1979); Kingsman et al., Gene 7:141 (1979); Tschumper et al., Gene 10:157 (1980)) and the plasmid YEp13 (Bwach et al., Gene 8:121-133 (1979)) are used, for example. The plasmid YRp7 contains the TRP1-gene which provides a selection marker for a yeast mutant (e.g ATCC No. 44076) which is incapable of growing in tryptophan-free medium. The presence of the TRP1 defect as a characteristic of the yeast strain used then constitutes an effective aid to detecting transformation when cultivation is carried out without tryptophan. The same is true with the plasmid YEp13, which contains the yeast gene LEU-2, which can be used to complete a LEU-2-minus mutant.

Other suitable marker genes for yeast include, for example, the URA3- and HIS3-gene. Preferably, yeast hybrid vectors also contain a replication start and a marker gene for a bacterial host, particularly E. coli, so that the construction and cloning of the hybrid vectors and their precursors can be carried out in a bacterial host. Other expression control sequences suitable for expression in yeast include, for example, those of PHO3- or PHO5-gene.

Other suitable promoter sequences for yeast vectors contain the 5′-flanking region of the genes of ADH I (Ammerer, Methods of Enzymology 101:192-210 (1983)), 3-phosphoglycerate kinase (Hitzeman et al., J Biol. Chem. 255:2073 (1980)) or other glycolytic enzymes (Kawaski and Fraenkel, BBRC 108:1107-1112 (1982)) such as enolase, glycerinaldehyde-3-phosphate-dehydrogenase, hexokinase, pyruvate-decarboxylase, phosphofructokinase, glucose-6-phosphate isomerase, phosphoglucose-isomerase and glucokinase. When constructing suitable expression plasmids, the termination sequences associated with these genes may also be inserted in the expression vector at the 3′-end of the sequence to be expressed, in order to enable polyadenylation and termination of the mRNA.

Other promoters are the promoter regions of the genes for alcohol dehydrogenase-2, isocytochrome C, acid phosphatase and enzymes which are responsible for the metabolism of altose and galactose. Promoters which are regulated by the yeast mating type locus, such as promoters of the genes BARI, MFα1, STE2, STE3, STE5 can be inserted in temperature regulated systems by the use of temperature-dependent sir mutations. (Rhine, Ph.D. Thesis, University of Oregon, Eugene, Oreg. (1979); Herskowitz and Oshima, The Molecular Biology of the Yeast Saccharomyces, Cold Spring Harbor Laboratory Press, part I, pp. 181-209 (1981)). Generally, however, any vector which contains a yeast-compatible promoter and origin replication and termination sequences is suitable. Thus, hybrid vectors which contain sequences homologous to the yeast 2μ plasmid DNA may also be used. Such hybrid vectors are incorporated by recombination within the cells of existing 2μ-plasmids or replicate autonomously.

In addition to yeasts, other eukaryotic systems may, of course, be used to express the polypeptides according to the invention. Since post-translational modifications such as disulphide bridge formation, glycosylation, phosphorylation and/or oligomerization are frequently necessary for the expression of biologically active eukaryotic proteins by means of recombinant DNA, it may be desirable to express the DNA according to the invention not only in mammalian cell lines but also insect cell lines.

Functional prerequisites of the corresponding vector systems comprise, in particular, suitable promoter, termination and polyadenylation signals as well as elements which make it possible to carry out replication and selection in mammalian cell lines. For expression of the DNA molecules according to the invention it is particularly desirable to use vectors which are replicable both in mammalian cells and also in prokaryotes such as E. coli.

Vectors derived from viral systems such as SV40, Epstein-Barr-virus, etc., include, for example, pTK2, pSV2-dhfv, pRSV-neo, pKO-neo, pHyg, p205, pHFBo, etc. (Sambrook et al., Molecular Cloning: A Laboratory Manual, Cold Spring Harbor Press, N.Y. (1989)).

After transformation in suitable host cells, e.g. CHO-cells, corresponding transformed cells may be obtained with the aid of selectable markers (thymidine-kinase, dihydrofolate-reductase etc.) and the corresponding polypeptides are isolated after expression. The host cells suitable for the vectors are known, as are the techniques for transformation (micro-injection, electroporation, calcium phosphate method, etc.) as described, for example, in Sambrook et al., Molecular Cloning: A Laboratory Manual, Cold Spring Harbor Press, N.Y. (1989).

For cloning corresponding DNA fragments in prokaryotic or eukaryotic systems, the selected vector may cut, for example, with a restriction endonuclease and, optionally after modification of the linearized vector thus formed, an expression control sequence equipped with corresponding restriction ends is inserted. At the 3′-end (in the direction of translation) the expression control sequence contains the recognition sequence of a restriction endonuclease, so that the vector already containing the expression control sequence is digested with the said restriction enzyme and the DNA molecule according to the invention, provided with ends which fit, can be inserted. It is advantageous to cleave the vector which already contains the expression control sequence with a second restriction endonuclease inside the vector DNA and to insert the DNA molecule provided with the correct ends into the vector fragment produced. The techniques required are described, for example, by Sambrook et al. Molecular Cloning: A Laboratory Manual Cold Spring Harbor Press. N.Y. (1989).

Apart from the DNA molecules specified, the invention also relates to processes for preparing the vectors described herein, particularly expression vectors. These vectors are characterized in that a DNA provided with corresponding ends and coding for a functional derivative or a fragment of the VLDL receptor is inserted into a vector DNA cut with restriction endonucleases and containing the expression control sequences described by way of example, in such a way that the expression control sequences regulate the expression of the DNA inserted. The peptides and antibody agents of the present invention which are obtained by the expression of recombinant DNA or from the native receptor molecule may, of course, also be derivatized by chemical or enzymatic processes.

The present invention is described in further detail in the following non-limiting examples.

### EXAMPLES

### Example 1

### Interaction of VLDL-R with APP

The interaction of very low density lipoprotein receptor (VLDL-R) with amyloid precursor protein (APP) was investigated.

VLDL-R was tagged with green fluorescent protein (VLDLRGFP) and the amyloid precursor proteins APP770 and APP695 were tagged with a MYC tag (APP770myc and APP695myc, respectively).

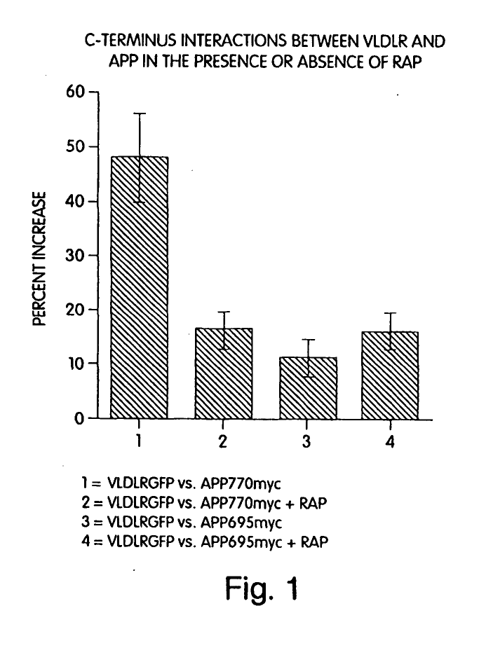

The proteins were combined with or without the presence of receptor-associated protein (RAP), and fluorescence energy transfer was assayed. The results of this assay are shown in FIG. 1.

The increase in fluorescence shown for the combination of VLDLRGFP and APP770myc represents close intramolecular association between VLDL-R and APP770. The amount of fluorescence resonance energy transfer approaches 50% between these proteins, suggesting a very strong and close intramolecular association.

In the presence of RAP, the interaction of VLDL-R and APP 770 was inhibited. Moreover, use of APP695myc, which does not contain the Kunitz protease inhibitor interacting domain on the amyloid precursor protein, also has markedly diminished interaction with VLDL-R. This implies that the protein domain on the amyloid precursor protein that interacts with ligand-binding domains on VLDL-R is the Kunitz protease inhibitor domain.

### Example 2

### RAP Reduces Aβ Production in LRP-Deficient Chinese Hamster Ovary Cells Transfected with APP751

CHO 13-5-1 are cell lines that were developed to be deficient in LRP (Fitzgerald, et al., 1995, J. Cell Biol. 129:1533-1541). These cells were stably transfected with APP751, and the effect of RAP on Aβ production assessed. In these experiments, the cells were plated in a 100 mm dish, and were cultured for four days in the presence (+RAP) or absence (control) of 500 nM RAP. Following this period, the media was removed, and replaced with serum-free media with or without 500 nM RAP, and the cells incubated for 24 h at 37° C. The media was then collected, and an aliquot assayed for total Aβ content using a commercial ELISA assay. The data were normalized to cell counts, and each experiment represents the average of five determinations.

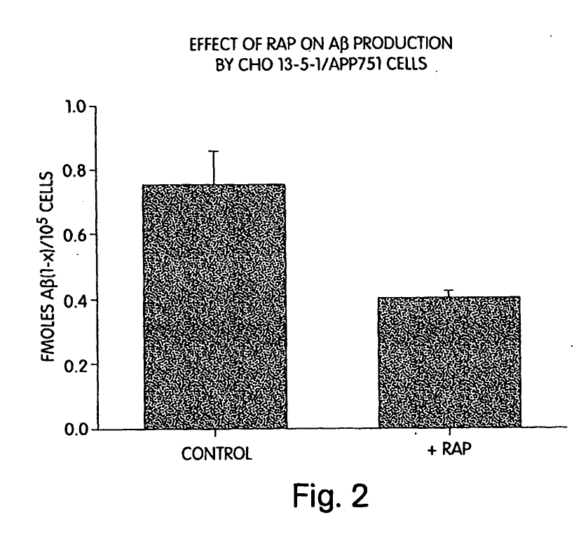

The results, shown in FIG. 2, demonstrate that RAP treatment reduces the amount of Aβ produced by the cells. This data reveals that a RAP-sensitive receptor, other than LRP, exists in CHO 13-5-1 cell lines. Further, like LRP, this RAP sensitive receptor can modulate APP processing such that Aβ levels are impacted. RAP ligand blotting experiments confirmed that the only other LDL receptor family member in these cell lines appears to be the VLDL receptor (Battey, et al., 1994, J Bio. Chem. 269:23268-23273), suggesting that the VLDL receptor may be responsible for this effect.

### Example 3

### Anti-VLDL Receptor IgG Reduces the Clearance of APP from the Cell Surface

To determine if the VLDL receptor alters the internalization rate of APP, the effect of blocking antibodies on the internalization of APP from the cell surface in PS70 cells was investigated. Three antibodies were used in these experiments. The first antibody was raised against a C-terminal peptide present in the LRP cytoplasmic domain. This antibody was used as a control, as this region of the molecule is inaccessible for antibody recognition in intact cells. The second antibody is an anti-LRP IgG that binds to the ligand binding domains of LRP and prevents ligands from binding to this receptor. The third antibody (anti-VLDL receptor IgG) is specific for the VLDL receptor, and blocks ligand recognition by this receptor.

PS70 cells were incubated with anti-LRP C-terminal IgG as a control, or blocking antibodies against LRP or the VLDL receptor for 1 h at 37° C. The cells were then chilled to 4° C., and anti-APP IgG added. Following incubation and washing, the cells were raised to 37° C., and the internalization of cell surface APP measured by measuring the decrease in anti-APP IgG on the cell surface.

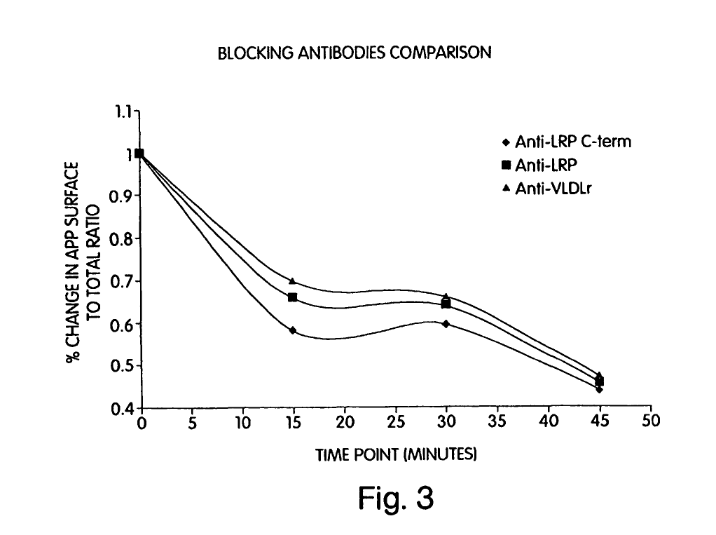

The results of this experiment are shown in FIG. 3, and demonstrate that both the LRP and the VLDL receptor antibodies delay the internalization of APP751 from the cell surface. These results indicate that APP interacts with LRP as well as the VLDL receptor on the cell surface, and that this interaction increases the internalization of APP.

### Example 4

### APP751 Binds to the VLDL Receptor

We next designed experiments to determine if the VLDL receptor directly binds to APP. To accomplish this, we performed binding experiments employing a soluble APP751 molecule fused to the Fc portion of IgG (sAPP-Fc). The sAPP-Fc molecule was prepared by transfecting cells with a plasmid containing the sAPP-Fc construct. Conditioned media from transfected or mock transfected cells was collected, and incubated with purified LRP or a soluble form of the VLDL receptor (prepared as described in Hembrough, et al., 2001, J. Biol. Chem. 276:12241-12248) immobilized on microtiter wells.

LRP (3 μg/ml) or sVLDL receptor (3 μg/ml) was coated on microtiter wells overnight at 4° C. Following coating, the wells were blocked with 3% BSA. Media from cells transfected with sAPP751-Fc was then added to the wells, and following incubation, the amount binding detected with an anti-APP IgG. As a control, media from mock transfected cells was also used, and no binding of this media was detected (data not shown).

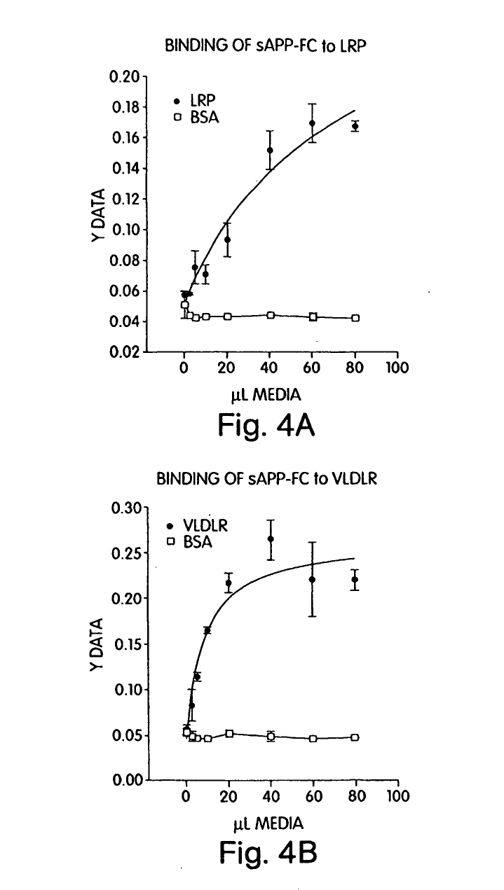

The results of this experiment are shown in FIG. 4, and demonstrate binding of sAPP-Fc to LRP (FIG. 4A) and to the VLDL receptor (FIG. 4B), but not to BSA. Media from mock transfected cells gave no signal in this assay. The interaction of sAPP751 with LRP is well known (Kounnas, et al., 1995, Cell 82:331-340), but these results for the first time document the interaction of sAPP to the VLDL receptor.

### Example 5

### ¹²⁵I-Labeled APP770 Degradation by VLDL-R-Expressing Cells

VLDL-R-expressing cells or VLDL-R-deficient cells are incubated with various concentrations of ¹²⁵I-APP770 or ¹²⁵I-APP695 for 10 h at 37° C. in the absence of competitor or in the presence of RAP. ¹²⁵I-APP770 is degraded by VLDL-R-expressing cells and not by VLDL-R-deficient cells. ¹²⁵I-APP695 is not degraded to any significant degree by VLDL-R-expressing cells or VLDL-R-deficient cells.

### Example 6

### Time Course of the cellular Internalization and Degradation of ¹²⁵I-APP770

VLDL-R-expressing cells and VLDL-R-deficient cells are incubated for selected times with ¹²⁵I-APP770 in the absence of RAP or in the presence of RAP. The amount of radioactivity internalized and degraded is determined at the selected time intervals. The inclusion of RAP significantly inhibits the internalization and degradation of ¹²⁵I-APP770 in VLDL-R-expressing cells but not in VLDL-R-deficient cells.

### Example 7

### VLDL-R Antibodies Inhibit the Degradation of APP770

VLDL-R-expressing cells are incubated for 18 h at 37° C. with ¹²⁵I-APP770 in the absence of competitor or in the presence of either RAP, affinity purified anti-VLDL-R IgG, or control IgG. The degradation of APP770 is inhibited by RAP and anti-VLDL-R antibodies but not control antibodies.

### Example 8

### Inhibition of APP/VLDL-R Interaction by RAP Fragments and Anti-VLDL-R Antibody Fragments

Fluorescence energy transfer assays of the interaction of VLDLRGFP with APP770myc and APP695myc are performed as described in Example 1.

The proteins are combined with various fragments of RAP and anti-VLDL-R antibodies, and fluorescence energy transfer is assayed. Fragments of RAP and anti-VLDL-R antibodies that inhibit the interaction of VLDL-R with APP are thereby identified.

Equivalents

All references disclosed herein are incorporated by reference.

Those skilled in the art will recognize, or be able to ascertain using no more than routine experimentation, many equivalents to the specific embodiments of the invention described herein. Such equivalents are intended to be encompassed by the following claims.

We claim:

1. A method for reducing catabolism of extracellular secreted amyloid precursor protein comprising
contacting a mammalian cell with an agent that reduces the amount or rate of binding of amyloid precursor protein (APP) with the very low density lipoprotein receptor (VLDL-R).

2. The method according to claim 1, wherein the agent is an agent which binds to the amyloid precursor protein.

3. The method according to claim 2, wherein the agent is an antibody or an antibody fragment containing the antigen binding domain that binds to amyloid precursor protein.

4. The method according to claim 2, wherein the agent is a functional derivative or fragment of VLDL-R.

5. The method according to claim 1, wherein the agent is an agent that binds to VLDL-R.

6. The method according to claim 5, wherein the agent is an antibody or an antibody fragment containing the antigen binding domain that binds to VLDL-R.

7. The method according to claim 5, wherein the agent is 39 kDa receptor associated protein (RAP) or a RAP fragment containing the binding domain that binds to VLDL-R.

8. The method according to claim 5, wherein the agent is functional derivative or fragment of amyloid precursor protein that binds to VLDL-R.

9. The method according to claim 1, wherein said contacting occurs in vitro.

10. A method for reducing the rate of onset or the severity of Alzheimer's disease, comprising
administering to an animal one or more agents that bind to the APP-binding site on VLDL-R (Group I agents) and/or one or more agents that bind to the VLDL-R-binding site found on APP (Group II agents) in an amount effective to reduce the rate of APP binding to VLDL-R.

11. The method according to claim 10, wherein the agent is an agent which binds to the amyloid precursor protein.

12. The method according to claim 11, wherein the agent is an antibody or an antibody fragment containing the antigen binding domain that binds to amyloid precursor protein.

13. The method according to claim 11, wherein the agent is a functional derivative or fragment of VLDL-R.

14. The method according to claim 10, wherein the agent is an agent that binds to VLDL-R.

15. The method according to claim 14, wherein the agent is an antibody or an antibody fragment containing the antigen binding domain that binds to VLDL-R.

16. The method according to claim 14, wherein the agent is 39 kDa receptor associated protein (RAP) or a RAP fragment containing the binding domain that binds to VLDL-R.

17. The method according to claim 14, wherein the agent is functional derivative or fragment of amyloid precursor protein that binds to VLDL-R.

18. The method according to claim 10, wherein the animal is a human.

19. A pharmaceutical composition comprising
one or more agents that bind to the APP-binding site on VLDL-R (Group I agents) and one or more agents that bind to the VLDL-R-binding site found on APP (Group II agents), and
a pharmaceutically acceptable carrier.

20. The pharmaceutical composition according to claim 19, wherein the agent which binds to the amyloid precursor protein is a functional derivative or fragment of VLDL-R.

21. The pharmaceutical composition according to claim 19, wherein the agent that binds to the APP-binding site on VLDL-R is an antibody or an antibody fragment containing an antigen binding domain that binds to VLDL-R.

22. The pharmaceutical composition according to claim 19, wherein the agent that binds to the APP-binding site on VLDL-R is 39 kDa receptor associated protein (RAP) or a RAP fragment containing the binding domain that binds to VLDL-R.

23. The pharmaceutical composition according to claim 19, wherein the agent that binds to the APP-binding site on VLDL-R is functional derivative or fragment of amyloid precursor protein that binds to VLDL-R.

24. A pharmaceutical composition comprising
a functional derivative or fragment of VLDL-R that binds to the amyloid precursor protein, and
a pharmaceutically acceptable carrier.

25. A pharmaceutical composition comprising
one or more agents that bind to the APP-binding site on VLDL-R (Group I agents)

26. The pharmaceutical composition according to claim 25, wherein the agent that binds to the APP-binding site on VLDL-R is an antibody or an antibody fragment containing an antigen binding domain that binds to VLDL-R.

27. The pharmaceutical composition according to claim 25, wherein the agent that binds to the APP-binding site on VLDL-R is 39 kDa receptor associated protein (RAP) or a RAP fragment containing the binding domain that binds to VLDL-R.

28. The pharmaceutical composition according to claim 25, wherein the agent that binds to the APP-binding site on VLDL-R is functional derivative or fragment of amyloid precursor protein that binds to VLDL-R.
